(19) United States
(12) Patent Application Publication
(10) Pub. No.: US 2005/0287195 A1
(43) Pub. Date: Dec. 29, 2005

(54) Transdermal drug delivery systems

(76) Inventors: Montague Cecil Solomon, London (GB); Biljana Tocili, London (GB); David Louis Charles Solomon, London (GB)

Correspondence Address: Martin Faier;Faier & Faier P.C., 566 W. Adams St. #600, Chicago, IL, 60661, US

(21) Appl. No.: 10/470,633

(22) Filed: Dec. 1, 2003

Related U.S. Application Data
(63) Continuation of application No. PCT/GB99/03811, filed on Nov. 17, 1999.
(63) Continuation-in-part of application No. 09/868,882, filed on Oct. 1, 2001, now abandoned.

(30) Foreign Application Priority Data
Dec. 24, 1998 (GB) GB98288480
Dec. 24, 1998 (GB) 9828480.5

(51) Int. Cl. A61K 9/70

(52) U.S. Cl. 424/449

(57) ABSTRACT

In a method of manufacturing such a system, an active substance is dissolved in a ratio less than saturation level in a solvent which is also a skin penetration enhancer. The system is coated as a layer onto a siliconized release paper and laminated onto a backing strip.

This application is a Continuation-in-Part of U.S. Ser. No. 09/868,882 filed on 1 Oct. 2001, which was a national phase entry of PCT/GB99/03811 dated 17 Nov. 1999, which claimed priority from GB 98288480 dated 24 Dec. 1998.

### TECHNICAL FIELD

The invention relates to transdermal drug delivery systems, that is systems for the administration of medicine through the skin of a patient and into the systemic circulation system. In this way, the medicine avoids passing through the gastro-intestinal tract and liver. Thus metabolism is to some extent avoided, and a smaller dose can be used.

### BACKGROUND ART

GB 2249956 contains a useful summary of the state of the art, and discloses such systems containing super-saturated solutions of an active ingredient within an adhesive layer by use of a carefully-selected mixture of solvents.

### THE INVENTION

As a generality, known methods for the manufacture of transdermal drug delivery systems comprise dispersing dissolving the pharmaceutically active substance in a carrier, penetration enhancer or optionally a solvent or mixture of solvents, adding an adhesive to create a thicker spreading composition and spreading the composition to create a film which is then dried. Fick's Law dictates that the pharmaceutically active substance is presented in a saturated or suspersaturated concentration, because that is the condition which was always believed to give the best transdermal transport rate of the drug. The addition of one or more skin penetration enhancers to the mixture is to improve the transdermal transport of the drug.

It has been suggested in EP-A-276561 that the pharmaceutically active substance piroxiam at a particular pH can be present at less than saturation concentrations without loss of the transdermal transport characteristics that epitomise a good transdermal drug delivery system. The present invention provides for effective transdermal delivery of any number of pharmaceutically active substances at less than the saturation concentrations. The invention selects, as a solvent to create the original solution of the pharmaceutically active substance, a solvent that is both a good solvent for the drug and a good skin penetration enchanter, and thus the creation of the initial solution of the active substance is made easier because the use of a separate aqueous or organic solvent is unnecessary. Because of the skin penetration enhancement properties of the solvent, good transdermal transport properties can be achieved even at subsaturated drug concentrations. Indeed the transdermal drug transport characteristics from subsaturated solutions have been found to be even better than those at saturated or supersaturated concentrations, which is contrary to what is known in the art, as exemplified by Fick's Low.

Investigations by the inventors found that two skin penetration enchancers in particular showed sufficiently high dissolution properties for a range of hydrophilic and hydrophobic drugs to be used as the solvent to create the original solution of the active substance. These solvent/enhancers are crotamiton and diethyltoluamide (DEET).

Furthermore, experiments by the inventors showed that the greatest and most unexpected advantage of a formulation method which (a) uses crotamiton and/or DEET as the solvent to create the initial solution of the drug and (b) creates that solution at subsaturation concentration lies in the subsequent control of the drying temperature after the drug solution is mixed with the adhesive, spread into a film and dried. If the drying conditions are such as to maintain the drug in a subsaturated concentration, then it is possible to avoid the formation of crystalline deposits of the drug in the adhesive layer of the transdermal drug delivery system. This has been found significantly to improve the consistency of the transdermal transport dynamics when the drug delivery system is used on a patent that improvement was not to be expected, because it had previously been thought that use presence of crystalline deposits in the adhesive layer was, desirable, establishing a reservoir of drug to be released first into saturated solution in the adhesive layer and then through the skin of the patient. For that reason drying conditions have never been believed to be significant, and are seldom if ever reported.

The present invention draws together all of the above of observations, and establishes a method for the manufacture of a transdermal drug delivery system which is economical and which provides a delivery system that delivers the active substance transdermally at high, regular and sustainable delivery rates even after the manufactured system has been allowed to stand on a shelf for prolonged periods of up to two years.

The invention provides a method for the manufacture of a transdermal drug delivery system, which comprises the successive steps of:
(a) dissolving a pharmaceutically active substance in one or both of the skin penetration enhancers crotamitan and diethyltoluamide (DEET) as solvent, to form a solution of active substance in the sin penetration enhancer(s) at a concentration less than saturation;
(b) mixing the resulting solution with an adhesive in the form of a solution or an aqueous dispersion;
(c) forming the mixture obtained in (b) into a film on a release liner or backing sheet, and
(d) drying the film at a temperature less than the boiling point of the skin penetration enhancer or enhancers used as solvent in the dissolution step (a) to maintain the active substance in solution at a concentration sufficently less than the saturation concentration that the product does not experience crystallisation of the active substance from the solution both during processing and later throughout the shelf life of the product.

By using the active substance in a ratio less than saturation level, there is a reduced risk of crystallisation, a stable system can be manufactured, and a reproducible constant rate of delivery to the patient can be obtained.

It is surprising that certain solvents act both as a skin penetration enhancer and as a solvent for the active substance. Such solvent/enhancers are crotamiton, diethyltoluamide (DEET) and mixtures thereof. Pure crotamiton or pure DEET can be used as the solvent, but if a solvent mixture is desired then the ratio of crotamiton to DEET in such a solvent mixture may be from 5:95 to 95:5% by weight of the total solvent/enhancer content depending on the delivery rate and extent of the delivery required for the active substance. By choosing a solvent/enhancer or solvent/enhancers having a boiling point higher than any drying temperature applied to the system, and controlling the drying temperature, the solvent(s) do not evaporate, the solution of the active substance never becomes saturated, and a high proportion of active substance remains in the system. The solution from step (a) can be maintained at 20°-30° C. for over one month before being admixed in step (b) with the adhesive. During that time no crystalline deposits of the drug are formed, because the solution of the active substance in the crotamiton and/or DEET is subsaturated. That is a first commercial advantage over known Formulation methods in which a supersaturated solution of the active substance is used which on unintended nucleation (which is not always possible to avoid) can start to crystallise within hours and importantly before production is complete.

The pharmaceutically active substance may be:
α-Adrenergic agonists such as Adrafonil, Adrenolone, Amidephrine, Aprochonidine, Clonidine, Ephedrine, Naphasoline and Tramazoline; β-Adrenergic agonists such as Albuterol, Clenbuterol Clorprenaline, Methoxyphenamine and Terbuterol;
α-Adrenergic blockers such as Amosulalol, Dapiprasol, Ergoloid Mesylates, Prazosin, Terzosin, Yohimbine;
β-Adrenergic blockers such as Acebutolol, Alprenolol, Atenolol, Pindolol, Propanolol and Timolol;
Anablics such Androstenediol, Ethylstrenol, Methandriol, Nandrolone, Oxzymesterone, Quinbolone and Stenbolone;
Analgesic (narcotic) such as Alfentanil, Benzylmorphine, Buprenorphine, Codeine, Codeine Phosphate, Dihydrocodeine, Dihydromorphine, Fentanyl, Methadone Hydrochloride, Morphine, Morphine Derivatives, Narceine, Opium, Oxycodone, Oxymorphone, Phenazocine and Sufentanil;
Analgesics (non-narcotic) such as Acetaminophen, Acetanilide, Acetylsalicylic Acid, Carbamazepine, Diflunisal, Indomethacin, Ketoprofen, Naproxen, Phenacetin, Salicylamide and Tramadol;
Androgens such as Mesterolone, 17-Methylestosterone, Testosterone and Testosterone and Testosterone Propionate;
Anaesthetics such as Amylocaine Hydrochloride, Bupivacaine, Lidocaine, Midazolam, Procaine, Tetracaine Hydrochloride, Thiopental Sodium and Zolamine;
Anti-acne drugs such as Algestone Acetophenide, Benzoyl Peroxide, Cyproterone, Resorcinal, Retinoic Acid and Tetroquinolone;
Anti-amebic such as Chloroquine, Chlortetracyline, Dehydroaemetine, Emetine, Teclosan, Thiocarbamazine, and Tinidazole;
Antianginals such as Alpreholol, Amlodipin Dilitiazem, Felodipine, Isosorbide Dinitrate, Nicardipine, Nifedipine, Nitroglycerin, Oxprenolol, Pindolol, Timoloil and Verapamil;
Antibacterial drugs such as Gentamiocin, Kanamycin, Neomycin, Chloramphenicol, Chloramphenicol Pantothenate, Clindamycin, Lincomycin, Clarithromycin, Erthromycin and Clycoserine;
Anti-estrogens such as Delmadinone Acetate, Tamoxifen and Toremifene;
Antifungal drugs such as Clotrimazole, Econazole, Ketocanazole, Miconazole and Potassium Iodide;
Antihistamines such as Chlorpheniramine, Dimethindene, Pheniramine, Triprolidine and Phenothiazine;
Antihyperetensive drugs such as Captopril, Enlalpril, Chonidine and Minoxidil;
Anti-inflammatory drugs such as Mefenamic Acid, Flubiprofen Ibuprofen, Indomethin, Ketoprofen, Aspirin, Mesalamine, Olsalasine, Piroxicam and Tenoxicam;
Anti-parkinsonian drugs such as Amtadine, Levodopa, Pergolide and Pridinol;
Antipyretics such as Camphor, Menthol, Phenol, Polidocanol, Spirit of Camphor and Trimeprazine;
Anti-seborrheic drugs such as Pyrithione, Resorcinol, Selenium sulphides and Tioxolone;
Antisepctics such as Chlorhexidine, Bismuth, Lodide Oxide, Povidone Iodine, Triclosan, Triclosane Potassium, Carvacrol, p-Cresol, Chloroxine, Halquinol, Boric Acid, α-Terpineol and Chlorhexidine;
Anti-ulcerative drugs such as Cimetidine, Enprostil, Omeprasol, Ranitidine and Trimoprostil;
Anxiolytic drugs such as Buspirone, Bromazepam, Diazepam, Loxapine, and Meprobamate;
Chlorineregic agents such as Bethanechol Chloride, Physostigmine and Pyridostigmine Bromide;
Depigmentors such as Hydroquinine, Hydroquinone and Monobenzone;
Estrogens such as Benzestrol, Dienestrol, Diethylstilbestrol, Dimestrol, Methestrol, Conjugated estrogenic Hormones, Equilenin, Equilin, Estradiol, EstradiolBenzoae, Estradiol 17β-Cypionate, Estriol, Estrone, Ethinyl Estradiol, Mestranol, Moxestrol, Quinestradiol and Quinestrol;
Gonadotropic hormones such as LH and PMSG;
Nootropic agents such as Aceglutamide, Antiracetam, Piracetam, Pyritinol and Tacrine.
Progestogens such as Allylestrenol, Anagestone, Chlormadinone Acetate, Delmadinone Acetate, Demegestone, Desogestrel, Dimethisterone, Dydogesterone, Ethisterone, Ethynodiol, Flurogestone Acetate, Gestodene, Gestodene Caprolate, Haloprogesterone, 17-Hydroxy-16-methylene-progesterone, 17α-Hydroxyprogesterone, 17-α-Hydroxygesterone Caprolate, Lynestrenol, Medrogestone, Medroxyprogesterone, Megestrol Acetate, Melengestrol, Norethisterone, Norethisterone Acetate, Noretynodrel, Norgesterone, Norgesimate, Norgestrel, Norgestienone, Norvinistyerone. Pentagestrone, Progesterone, Promegestrone, Quingestrone and Trengestone;
Respiratory stimulants such as Almitrine, Doxapram, Lobeline, Sodium Succinate and Tacrine;
Vitamins, vitamin sources and vitamin extacts such as Vitamins A, B, C, D, E and K and derivatives thereof, Calciferols, Glycyrrhiza and Mecobalamin; or
Vulnerary agents such as Acetylcysteine, Allantoin, Asiaticoside, Cadexomer Iodine, Chitin, Dextranomer and Oxaceprol.

The solvent/enhancer is Crotamiton and/or Diethyltoluamide (DEET). If desired the solution obtained in step (a) can also incorporate at least one antioxidant, for example butylhydroxytoluene (BHT), and/or at least one other skin penetration enhancer, preferably selected from the group consisting of Transcutol (Diethylene glycol monoethyl ether), Labrafil M1944CS (unsaturated polyglycolysed glycerides), Labrasol (Glyceryl and pllyethylene glycol esters), Tea-tree oil (Oil of Melaleuce). Propylene Glycol, MP DIOL (2-Methyl-1,3-Propanediol) and Polyethelyne Glycol.

It will be appreciated that the amount of active substance to be incorporated in the delivery system is dependent or governed by the drug composition and/or concentration, the desired therapeutic effect for a patient, and the period for which the delivery system is to provide a therapeutic drug level. Preferably, the active substance is present in an amount from 0.1% to 50% by weight of the coating material (i.e. an aqueous emulsion or adhesive solution). More preferably, 0.3% to 30% by weight of the coating material.

The active substance is generally incorporated in the solvent/enhancer at room temperature (25° C. or below) and in a ratio less than 90% of saturation level to prevent crystal formation during storage. Dissolution may be aided by sonication or warming. The resulting solution can be added slowly to the adhesive which may be in the form of an aqueous dispersion or a solution and mixed. The adhesive can be an acrylate, silicone or polyisobutylene. An adhesive thickener may be added to the mixture at about 50% solution/water mix to produce a thicker spreading solution for reverse roll coating or knife over roll coating.

The resulting delivery system may then be coated into a release liner, which may be a siliconised polyester such as type FL 2000 (commercially available), or siliconised paper, which naturally is impermeable to the active substance. The system can then be dried by circulating hot air, and laminated onto a backing sheet, which may be a 3M Health Care type 1220, the backing sheet generally being impermeable to the active substance. The layer may be coated to a wet-coat thickness of from 20 to 500 μm. Alternatively, the delivery system mixture may be spread or coated onto the backing sheet, and then laminated to the release liner. The hot air circulation may be effected at gradually increasing temperatures from 50° C. to 140° C., provided that the drying is carried out under conditions which maintain the active substance in solution at less than the saturation concentration.

If the active substance is dissolved in the crotamiton and/or DEET skin penetration enhancer at a concentration of less than 90% of saturation level, preferably less than 80% of saturation level, the resulting delivery system is found to have a shelf life of at least two years, without experiencing any crystallisation of the active substance. In comparison, similar transdermal delivery systems made by methods involving the creation of saturated or supersaturated solutions of the active substance have been found to experience crystal growth of the active substance in the film coating in substantially shorter periods. Conventional wisdom has been that the existence of crystals in the patch can be beneficial, as the crystals of active substances within the film provide a reservoir for the replenishment of active substances into the delivery system when the system is applied to the skin of a user and the active substance migrates transdermally from the saturated solution surrounding those crystals Into the skin of the user. The inventors believe that this common understanding of the mechanism of the delivery system is wrong. Active substances are delivered transdermally at a faster rate than the formed crystals can dissolve to replenish the system, so if crystallisation has taken place the vast majority of the active substance in crystal form is never delivered to the patient but remains in crystal form until the patch or other delivery device is removed and discarded. That is wasteful of active ingredient, which is often by far the most expensive component of the system. Also, the dosage delivery rates of the known systems are compromised by crystal formation, as the formation of crystals within the film reduces the concentration of the active ingredient remaining in solution in the film, so that no products will generally, after crystallisation has commenced, have exactly the same dosage delivery rates as the same products in the absence of crystallisation. This depends on the level of crystallisation, which is unpredictable once crystal growth has started. Most modern commercial medication systems require a shelf life of at least two yeas without loss of therapeutic activity per unit dosage form and reproducible delivery of the drug from each unit dosage form. The delivery systems manufactured according to the invention can reliably and repeatedly attain that goal. The prior art systems cannot guarantee that.

### DRAWING

FIG. 1 is section through an adhesive tape for application to the skin of a patient comprising a drug delivery system according to the invention. A delivery system comprising an active substance adhesive and solvent/skin penetration enhancer 4 is coated as a layer onto a siliconized release paper 2 and laminated onto a backing strip 6.

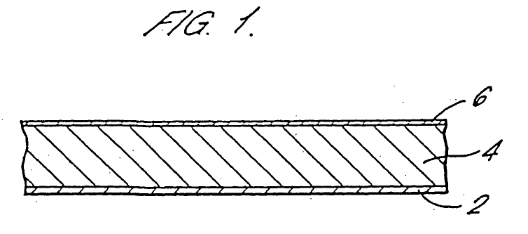

The following Examples of ingredients in parts by weight may be used in the production of delivery systems as described above:

| | | Ex. 1 | Ex. 2 | Ex. 3 | Ex. 4 | Ex. 5 | Ex. 6 | Ex. 7 | Ex. 8 | Ex. 9 | Ex. 10 |
|---|---|---|---|---|---|---|---|---|---|---|---|
| active sustance | Estradiol Hemihydrate | 1.0 | 1.0 | 1.0 | 1.0 | 0.9 | 0.9 | 0.9 | 1.2 | 1.1 | 1.0 |
| | Norethisterone Acetate | 2.4 | 2.4 | 2.4 | 2.4 | 2.4 | 2.4 | 2.4 | — | — | — |
| solvent/enhancer | DEET | — | — | 18.0 | 15.3 | 9.0 | 2.7 | 15.3 | — | 6.0 | 6.09 |
| | Crotamiton | 18.0 | 20.0 | — | 2.7 | 9.0 | 15.3 | 2.7 | 7.5 | 0.6 | — |
| other enhancer | Labrafil M (1944CS) | 4.25 | — | — | — | — | — | — | 2.0 | — | — |
| adhesive | Monsanto 3011 | 74.35 | — | — | — | — | — | — | — | — | — |
| | Monsanto 2484 | — | 76.6 | 78.6 | — | — | — | — | — | — | — |
| | Monsanto 2397 | | — | — | — | — | — | — | 89.3 | — | — |
| | C945/127 | — | — | — | 78.7 | 78.7 | 78.7 | — | — | — | 93.55 |
| | NS 2287 | — | — | — | — | — | — | 78.7 | — | 92.3 | — |
| | Acrysol ASE60 | — | — | — | — | — | — | — | — | — | 0.2-0.9 |
| | Ammonia BP (aq. dil) | qs | qs | qs | — | — | — | — | — | — | qs |
| | Purified water (BP) | qs | qs | qs | qs | qs | qs | — | qs | — | qs |

Manufacturing Method (Illustrative)
A) Delivery System Using Adhesive—Aqueous Emulsion

The active substance is dissolved in the solvent/enhancer by means of heating and mixing over a 45°-55° water bath with agitation. When the solution Is optically clear, it is checked microscopically for absence of crystals.

The adhesive is weighed into a separate mixing vessel, and diluted with water if necessary over a period not exceed 30 minutes to achieve the requisite viscosity. The active substance/solvent solution is gradually added to the adhesive with mixing. The pH is adjusted to 6.5-7.6 and a thickener is added (if appropriate) to obtain a suitable viscosity (eg 900-1000 cps) for the selected coating process such as reverse roll coating or knife over roll coating. It was found that this adhesive mix had a shelf life of over 24 hours, since over that period of lime there was no crystal growth.

The resultant mixture is coated onto a release liner (typical coating thickness 20-500 μm), and dried by passing in sequence through ovens at 50-140° C. Under these conditions the active substance remains in solution at less than saturation levels. The product is then laminated onto a backing sheet.

Storage Lifetime Tests

The absence of crystal growth over a prolonged storage period was observed in the products obtained according to the method of the invention and compared with commercially available transdermal patches, as follows. Two commercially available transdermal patches were purchased. The purchased product was a two-stage transdermal patch system sold under the Trade Mark Nuvelle TS. The two patches were intended to be used consecutively by a patient. One patch in the system contained estradiol as the active substance. The other used a combination of levonorgestrel and estradiol. Both patches are understood to have been made in accordance with the teachings of U.S. Pat. No. 5,352,457. The purchased products were left at 25° C. (the recommended storage temperature) over their stated shelf lives until their expiry dates. During that period they were checked at intervals both visually and microscopically for evidence of crystal growth.

Three separate batches of products made according to the invention were similarly inspected.

In no case was there any evidence of the growth of crystals within the coating of active substance in the products made according to the invention. One batch was monitored for sixteen months; another was monitored for eighteen months; and the third was monitored for thirty months. No crystal growth was discernible in any of the samples over any of those time periods.

In contrast, both patches from the commercially available products showed signs of crystal growth within their shelf life, the crystal clusters within the coating being clearly visible to the naked eye.

In-Vitro Drug Delivery Through the Skin

In vitro skin permeation experiments with human skin have been carried out on systems made in accordance with the above examples, using Franz-type diffusion cells, and the studies were carried out on a Hanson Microette system.

Dermatomed human skin sections were mounted onto the Franz cells and transdermal drug delivery systems mounted on tape backings (1.5 cm²) were placed on the stratum corneal surface of the skin. Each Franz cell contained 7 ml of ethanol phosphate buffered saline as the receptor medium, maintained at 32° C. At predetermined time intervals 0.7 ml of the receptor solution was sampled and an equal amount replaced. The samples were analysed by High Pressure Liquid Chromatography.

The skin mass transport of Estradiol; and Norethisterone Acetate has been found to be enhanced by the solvent/skin penetration enhancer DEET and/or Crotamiton in a concentration below saturation. Further the active substance flux profile follows the solvent flux profile, the latter showing high skin penetration flux during the first 20 hours of application.

Indications

The main indications for the HRT products are both peri-menopausal and menopausal women for the control in the former of the symptoms of the menopause such as hot flushes, sweating and other symptoms of the peri-menopause, and in the case of the menopause the prevention of osteroporosis and cardiac events such as coronary thrombosis. Other indications for other drugs are for example analgesia for fentanyl.

### EXAMPLES 11 AND 12

Transdermal patches were made in accordance with the invention as follows. Two different narcotic drugs were dissolved in a solvent/skin enhancer. When each solution was optically clear it was further checked under the microscope for the absence of any crystals in each case the solution was at that stage less than 90% saturated. In the case of Example 11 to the solvent/skin enhancer there was first added butylhydroxytoluene (BHT) in the stated amount. BHT is a known antioxidant and was included to ensure stability of the active substance over the probable shelf storage life of the resulting dermatological patches.

After formulation of the solution of the active substance in the skin penetration enhancer, both of the solutions of Examples 11 and 12 were made up into transdermal patches as described in relation to the preceding Examples. In both cases the drying temperatures were such that the active substances were maintained in subsaturated solution to an extent such that there was no crystallisation of the active substance in the final patch even after prolonged storage.

| | | Ex. 11 | Ex. 12 |
|---|---|---|---|
| active sustance | Fentanyl | 3.0 | — |
| | Buprenorphine | — | 1.2 |
| solvent/enhancer | DEET | 10.0 | 20.0 |
| | Crotamiton | — | — |
| other enhancer | Labrafil M (1944CS) | — | — |
| antioxidant | BHT | 1.0 | — |
| adhesive | Monsanto 3011 | — | — |
| | Monsanto 2484 | — | — |
| | Monsanto 2397 | — | — |
| | C945/127 | 85.7 | 78.5 |
| | NS 2287 | — | — |
| | Acrysol ASE60 | 0.3 | 0.3 |
| | Ammonia BP (aq. dil) | — | — |
| | Purified water (BP) | — | — |

1. A method for the manufacture of a transdermal drug delivery system, comprising the successive steps of:
(a) dissolving a pharmaceutically active substance in a skin penetration enhancer, selected from the group consisting of crotamiton, diethyltoluamide and a mixture thereof to form a solution having a less than saturation concentration of the active substance;
(b) mixing the step (a) solution with an adhesive in the form of a solution or an aqueous dispersion;
(c) forming a film of the step (b) mixture on a release liner or a backing sheet; and
(d) drying the step (c) film at a temperature less than the boiling point of the skin penetration enhancer to maintain a less than saturation concentration of the active substance in the skin penetration enhancer.

2. The method according to claim 1 wherein the solvent used in step (a) comprises crotamiton and diethyltoluamide in a weight percentage ratio of between about 5:95 to 95:5.

3. The method according to claim 1 wherein at least one other skin penetration enhancer is incorporated in the solution of step (a).

4. The method according to claim 3 wherein the other skin penetration enhancer is selected from the group consisting of diethylene glycol monoethyl ether, unsaturated polyglycolysed glycerides, glyceryl and polyethylene glycol esters, propylene glycol laurate, oil of Melaleuca, propylene glycol, 2-methyl-1,3-propanediol polyethylene glycol and any combination thereof.

5. The method according to claim 1 wherein the concentration of active substance in the skin penetration enhancer or enhancers in the step (a) solution is less than 90% of saturation.

6. The method according to claim 1 wherein the active substance is estradiol.

7. The method according to claim 1, further comprising adding an antioxidant to the skin penetration enhancer in step (a).

8. The method according to claim 1, further comprising adding an antioxidant to the skin penetration enhancer in step (a), and wherein the active ingredient is fentanyl.

9. The method according to claim 1 wherein the active substance is buprenorphine.

10. The method according to claim 1 wherein the adhesive is selected from the group consisting of acrylate, polyisobutylene and silicone adhesives.

11. The method according to claim 1 wherein the film is formed on a release liner, dried according to step (d), then laminated onto a backing sheet.

12. The method according to claim 1 wherein the film is formed on a backing sheet, dried according to step (d), then laminated onto a release liner.

13. The method according to claim 1 wherein the drying temperature in step (d) is increased gradually from 50° C. to 140° C.

14. The method according to claim 1, wherein the concentration of the active substance is sufficently less than saturation concentration to avoid crystallization during processing.

15. The method according to claim 1, wherein, after drying, the concentration of the active substance is sufficiently less than saturation concentration to avoid crystallization during the shelf-life of the product.

16. A transdermal drug delivery system comprising a pharmaceutically effective film formed between a backing sheet and a release liner, wherein the film comprises
(a) a pharmaceutically active substance;
(b) a skin penetration enhancer, selected from the group consisting of crotamiton, diethyltoluamide and a mixture thereof; and
(c) an adhesive,
wherein the concentration of active substance in the skin penetration enhancer is less than the saturation concentration.

17. The system according to claim 16, wherein the concentration of the active substance is sufficiently less than the saturation concentration to avoid crystallization throughout the shelf-life of the product.
